(19) United States
(12) Patent Application Publication
(10) Pub. No.: US 2021/0010049 A1
(43) Pub. Date: Jan. 14, 2021

(54) METHOD FOR PRODUCING ASTAXANTHIN

(71) Applicants: NIPPON SUISAN KAISHA, LTD., Tokyo (JP); BIOGENIC CO., LTD., Tokyo (JP)

(72) Inventors: Hitoshi IZUMIDA, Tokyo (JP); Eiji OHASHI, Tokyo (JP); Toru NUMASAWA, Tokyo (JP)

(21) Appl. No.: 16/334,670

(22) PCT Filed: Sep. 14, 2017
(86) PCT No.: PCT/JP2017/033222

§ 371 Date: Mar. 19, 2019

(30) Foreign Application Priority Data
Sep. 21, 2016 (JP) 2016-183740

(51) Int. Cl. C12P 23/00

(52) U.S. Cl. CPC C12P 23/00

(57) ABSTRACT

Efficiency of production method for producing astaxanthin by culturing microalgae is improved. A method for producing astaxanthin in which astaxanthin is produced in inner of algae by culturing a microalga, wherein a light irradiation during a green stage culturing of the microalga is performed by using both a blue LED of peak wavelength from 420 to 500 nm and a red LED of peak wavelength from 620 to 690 nm, and having a ratio of photon flux density of the blue LED to the red LED to be 2:3 to 20:1. It is preferable that the photon flux density of the blue LED is from 5 to 200 μmol/m²/s, and the photon flux density of the red LED is from 5 to 200 μmol/m²/s.

### TECHNICAL FIELD

The present invention relates to an efficient method for producing astaxanthin. More particularly, relates to light irradiation during green stage culturing when culturing microalgae that produce astaxanthin.

### BACKGROUND

Astaxanthin is a type of red-orange carotenoid, and is a pigment contained in large quantity primarily in marine organisms such as crustaceans like shrimp, crab, and the like, salmon, salmon roe, sea bream, algae, and the like. Astaxanthin is known to have a powerful antioxidant action, and is used in food coloring, cosmetics, health foods, medicines, and the like.

Astaxanthin is produced by chemical synthesis or by culturing bacteria, yeasts, microalgae, and the like. The astaxanthin content in per dry weight of bacteria or yeast is not more than 2% by weight, whereas a high content that is not less than 2% by weight can be obtained by culturing microalgae of the Haematococcus genus (referred to as “Haematococcus” hereinafter), and due to its safety, it is produced worldwide. During producing astaxanthin using microalgae that perform photosynthesis such as Haematococcus and the like, light irradiation suitable for their growth is required.

Astaxanthin, for example, is produced by microalgae such as Haematococcus, Chlorella, Scenedesmus, and the like. In particular, due to stress caused by changes in the external environment, Haematococcus are encysted and accumulate astaxanthin inside. To accumulate astaxanthin, irradiation by sunlight or artificial light is required. Fluorescent lamps, light emitting diodes (LEDs), and the like are used as artificial light sources.

The microalgaes producing astaxanthin are known to have the following states: a planktonic cell state (i.e., green stage), which is green, and has zoospore with two flagellums, and has motility and intensified cell proliferation under a culturing condition of suitable light irradiation; and a state (i.e., red stage), in which the planktonic cells turn into cyst cells due to stress caused by temperature, intense light, salt, nutrient depletion, and the like, so as to accumulate astaxanthin in inner of cells. Astaxanthin is hardly contained in planktonic cell, but is accumulated at a high concentration during as cyst cells.

In the case of culturing using only sunlight, stable and efficient production is difficult to be performed because it is affected by fluctuations in air temperature and fluctuations in duration of daylight. Therefore, culturing using a fluorescent lamp which is one of artificial light source has been attempted since long ago.

LED is known as a light source with low power and low heat generation, and the production of astaxanthin using LED instead of fluorescent lamps has been researched.

The present inventors have reported in Patent Document 1 that in the case of producing astaxanthin by culturing microalgae, the production efficiency of astaxanthin can be improved by using both blue LED and red LED during red stage culturing after encystation.

Regarding the light irradiation during green stage culturing before encystation, the use of both blue light and red light according to relationship with absorption wavelength of chlorophyll has been researched, and is reported by Patent Documents 2˜5 and the like. All of them improved the growth of algae and increased the amount of algae (the quantity of planktonic cells) in culture solution by using both blue light and red light.

### PRIOR ART DOCUMENTS

### Patent Documents

Patent Document 1: WO2015/151577

Patent Document 2: WO2014/119789

Patent Document 3: WO2014/119792

Patent Document 4: WO2014/119794

Patent Document 5: CN102766578

### SUMMARY OF THE INVENTION

### Problems to be Solved by the Invention

The problem is to provide a method that can further improve the production efficiency of astaxanthin culturing method using LED with low power and low heat generation.

### Means for Solving the Problems

An object of the present invention is to use LED that save power and can suppress temperature rise of the portion transmitting light, so as to produce astaxanthin efficiently.

In Patent Documents 2 to 5, it is disclosed that the amount of algae in culture solution can be increased by irradiating with blue light and red light during green stage culturing, but there has been no research on whether the astaxanthin production quantity is also increased. Therefore, it is only disclosed that culturing is performed under a condition in which photon flux density of blue light is equal to photon flux density of red light or the photon flux density of red light is stronger.

However, the inventors of the present application have found that during culturing in green stage culture, the photon flux density of blue light with respect to red light is increased, whereby although the increasement of algae decreases, the accumulation of astaxanthin in algae increases, as a result, the production quantity of astaxanthin in culture solution increases, thereby the invention of the present application is completed.

As a result of diligent research to achieve above the objective, it is discovered that during culturing microalgae in green stage, increasing photon flux density of blue LED compared with the priority art, meanwhile irradiating with blue light LED of peak wavelength from 420 nm to 500 nm and red light LED of peak wavelength from 620 nm to 690 nm and performing culturing, such that astaxanthin can be effectively produced. In particular, continuous light irradiation is preferred. That is, the more quantity of light is irradiated, the more astaxanthin content in red stage can be obtained, and therefore, as a result, the production efficiency of astaxanthin is improved compared with alternate irradiation.

The gist of the present invention comprises the following methods (1) to (8) for producing astaxanthin.

(1) A method for producing astaxanthin in which astaxanthin is produced in inner of algae by culturing a microalgae, wherein a light irradiation during a green stage culturing of the microalgae is performed by using both a blue LED of peak wavelength from 420 to 500 nm and a red LED of peak wavelength from 620 to 690 nm, and having a ratio of photon flux density of the blue LED to the red LED to be 2:3 to 20:1.

(2) The method for producing astaxanthin according to (1), wherein the ratio of photon flux density of the blue LED to the red LED is 3:2 to 20:1.

(3) The method for producing astaxanthin according to (1) or (2), wherein the light irradiation using the blue LED and the red LED is continuously performed during the green stage culturing.

(4) The method for producing astaxanthin according to any one of claims 1) to (3), wherein the photon flux density of the blue LED is from 5 to 200 μmol/m²/s, and the photon flux density of the red LED is from 5 to 200 μmol/m²/s.

(5) The method for producing astaxanthin according to any one of (1) to (4), wherein the light irradiation during a red stage culturing of the microalgae is performed further using both the blue LED of peak wavelength from 420 to 500 nm and the red LED of peak wavelength from 620 to 690 nm.

(6) The method for producing astaxanthin according to (5), wherein the ratio of photon flux density of the blue LED to the red LED is 1:1 to 20:1 during the red stage culturing of the microalgae.

(7) The method for producing astaxanthin according to (5) or (6), wherein the photon flux density of the blue LED is from 20 to 1000 μmol/m²/s during the red stage culturing, the photon flux density of the red LED is from 20 to 1000 μmol/m²/s during the red stage culturing.

(8) The method for producing astaxanthin according to any one of (1) to (7), wherein the microalgae is Haematococcus genus.

### Effect of the Invention

According to the present invention, astaxanthin can be efficiently produced without significantly modifying conventional methods or/and equipments for producing astaxanthin.

### BRIEF DESCRIPTION OF THE DRAWINGS

FIG. 1 is a drawing illustrating spectra of blue LED and red LED used in an embodiment.

### MODE FOR CARRYING OUT THE INVENTION

The present invention relates to a method for producing astaxanthin using microalgae, wherein irradiate microalgae with a blue LED of peak wavelength from 420 to 500 nm and a red LED of peak wavelength from 620 to 690 nm at a ratio of photon flux density of the blue LED to the red LED to be 2:3 to 20:1, when microalgae is culturing in green stage (i.e., planktonic cell state which is green, and has zoospore with two flagellums, and has motility and intensified cell proliferation, before encystation).

In the present invention, a microalgae capable of producing astaxanthin can be used. The microalgae stated here is limited to those performing photosynthesis. It is known that microalgae include cyanobacteria, Rhodophyta, Phaeophyceae, Chlorophyceae, Bacillariophyceae, Eustigmatophyceae, and the like, but the microalgae of the present invention is limited to microalgae capable of producing astaxanthin. As microalgae producing astaxanthin, microalgae belonging to the Haematococcus genus (Haematococcus) are generally used.

As Haematococcus, Haematococcus lacustris, H. pluvialis, H. capensis, H. droebakensi, H. zimbabwiensis, and the like may be used. Among them, Haematococcus lacustris and Haematococcus pluvialis are preferably used.

In addition to Haematococcus genus, Microalgae producing astaxanthin may also be used. Examples may include microalgae of Chlorella zofingiensis, which is Chlorella genus, and Monoraphidium sp., as well as Vischeria helvetica, Coelastrella, Scenedesmus, Chlamydomonas nivalis, Protosiphon botryoides, Neochloris wimmeri, and the like.

The medium used in culturing of the microalgae is not particularly limited, but autotrophic medium containing no carbon source to prevent contamination of the medium by miscellaneous bacteria is preferred. An autotrophic medium containing nitrogen, inorganic metal salts of trace metals, vitamins, and the like required for proliferation is generally used. For example, media such as VT medium, C medium, MC medium, MBM medium, MDM medium, and the like (refer to Alga Research Methods, Nishizawa, K. and Chihara, M., Kyoritsu Shuppan (1979)), BG-11 medium, and modified media thereof are used.

Furthermore, when culturing microalgae in a medium, it is preferable to ventilate with air containing carbon dioxide. The microalgae may be cultured while ventilating with air containing no carbon dioxide, but since that retards the growth of the microalgae, they are cultured while ventilating with air containing from 0.1 to 5% carbon dioxide, and more preferably from 0.5 to 3% carbon dioxide. It is possible to culture the microalgae without ventilation, but for good development, the air flow rate is from 0.01 to 3.0 vvm and preferably from 0.015 to 1 vvm, in addition, the pH is from 5 to 10 and preferably from 6 to 9.

As for culturing temperature, taking the use of Haematococcus lacustris and Haematococcus pluvialis as an example, the culturing temperature is, for example, from 10 to 45° C. and preferably from 18 to 38° C. In addition, the pH of the medium is adjusted in the range from 5.0 to 9.5 and preferably in the range from 6.0 to 9.0.

Light irradiation of microalgae in green stage for producing astaxanthin is performed using both a blue LED of peak wavelength from 420 to 500 nm and a red LED of peak wavelength from 620 to 690 nm. Irradiation with both the blue LED and the red LED needs to be performed during the whole green stage culturing period, or during a certain green stage culturing period of microalgae. In the case that irradiation with both the blue LED and the red LED is performed, astaxanthin can be produced at the greatest efficiency by simultaneous irradiation, but astaxanthin can also be produced efficiently by alternately irradiating with the blue LED and the red LED within 24 hours. Alternatively, an irradiation method in which the blue LED and the red LED flash alternately may also be used. Intermittent irradiation having time interval can also be used. Here, “intermittent irradiation” includes irradiation with pulsed light. If light is intermittently irradiated, power consumption can be reduced. However, it is preferable that light irradiation is continuously performed for improving production efficiency. That is, the more quantity of light is irradiated, the more astaxanthin content in red stage can be obtained, and therefore, as a result, the production efficiency of astaxanthin is improved compared with alternative irradiation. Here, continuous irradiation dose not mean absolutely no interruption in 24 hours, but means that irradiation with blue light and the red light is performed for at least 12 hours in one day, preferably at least 15 hours, more preferably at least 18 hours or 21 hours, most preferably 24 hours in one day.

As the light source in the light irradiation step, an LED, an electric bulb, a fluorescent lamp, and the like may be used, but light sources other than LEDs have poor efficiency because light of the light sources has a wide wavelength spectrum, and therefore unnecessary light needs to be cut. If the LED is used, astaxanthin can be efficiently produced with low irradiation energy because irradiation of light having a narrow wavelength range is possible without requiring a special means to cut some of the light. An organic electro-luminescence lighting source may be used as the LED.

It is preferable to use a plurality of LED chips such that efficient irradiation is performed. If a plurality of light sources are used, it is preferable to dispose the light sources at equal intervals to enable as uniform light irradiation as possible. Furthermore, a plurality of chips of blue LED and red LED may independently be made into panels to irradiate, or irradiation may be performed using one panel embedded with a plurality of chips of blue LED and red LED in a certain proportion.

The irradiated wavelength of the blue LED has a peak wavelength from 420 to 500 nm and preferably from 430 to 490 nm, and the wavelength of the red LED is from 620 to 690 nm and preferably from 630 to 680 nm.

Each of blue LED and red LED may emit light of not less than two different peak wavelengths. For example, irradiation may be performed using blue LED of peak wavelengths 430 nm and 470 nm and red LED of peak wavelengths 630 nm and 660 nm.

Both blue LED and red LED preferably emit light having a narrow width of wavelength. This is because astaxanthin can be more efficiently produced by selecting only the light having a range of wavelength suitable for astaxanthin production to irradiate.

The ratio of the blue LED to the red LED that simultaneously irradiate microalgae by photon flux density during green stage culturing is from 2:3 to 20:1, and preferably from 1:1 to 20:1, or from 3:2 to 20:1, or from 3:2 to 10:1, or from 2:3 to 3:1, or from 2:1 to 10:1, or from 2:1 to 5:1, or from 2:1 to 4:1, and furthermore, particularly preferably from 2:3 to 5:1, or from 2:3 to 3:1, or from 2:1 to 3:1.

Haematococcus such as Haematococcus lacustris, Haematococcus pluvialis and the like take the form of the following states: a state of green planktonic cells having motility and intensified cell proliferation, and a state of cyst cells encysted due to stress from extreme changes in environmental conditions such as temperature, intense light, salt, moisture content, nutrients, and the like. Once encystation occurs, astaxanthin accumulates in the inner of the algal and the color turns to be red.

Light irradiation using the blue LED of peak wavelength from 420 to 500 nm and the red LED of peak wavelength from 620 to 690 nm may also be used in a state of cyst cells (i.e., red stage) in which astaxanthin accumulates in the inner of the cells. A natural light, a white light, a red light, and the like can be used in red stage, but using blue LED together with red LED is preferred. In this case, strengthening the blue light is more preferred (refer to Patent Document 1).

The planktonic cells having motility are numerous and the cells density is low in green stage of Haematococcus, therefore, they are proliferated well even at a photon flux density of not greater than 20 μmol/m²/s.

The photon flux density in green stage is not particularly limited as long as the cells proliferate without encystation or death, for example, as long as a culturing apparatus having a light transmission width (diameter, thickness) of not greater than 100 mm is used, astaxanthin can be efficiently produced by irradiation with blue LED of peak wavelength from 420 to 500 nm and red LED of peak wavelength from 620 to 690 nm, each having a photon flux density from 5 to 100 μmol/m²/s, preferably from 10 to 70 μmol/m²/s, and more preferably from 20 to 50 μmol/m²/s. Furthermore, as long as a culturing apparatus having a light transmission width (diameter, thickness) of not greater than 100˜400 mm is used, astaxanthin can be efficiently produced by irradiation with blue LED of peak wavelength from 420 to 500 nm and red LED of peak wavelength from 620 to 690 nm, each having a photon flux density from 10 to 200 μmol/m²/s, preferably from 20 to 150 μmol/m²/s, and more preferably from 30 to 100 μmol/m²/s.

The photon flux density is not particularly limited in red stage after encystation of Haematococcus by applying stress due to temperature, intense light, salt, and so on, for example, as long as a culturing apparatus having a light transmission width (diameter, thickness) of not greater than 70 mm is used, astaxanthin can be efficiently produced by irradiation with blue LED of peak wavelength from 420 to 500 nm and red LED of peak wavelength from 620 to 690 nm, each having a photon flux density of not less than 20 μmol/m²/s, preferably not less than 50 μmol/m²/s, and more preferably not less than 100 μmol/m²/s or not less than 150 μmol/m²/s or not less than 300 μmol/m²/s. If a culturing apparatus with a light transmission width greater than the above range is used, it may be even larger. That is, in the case that Haematococcus is cultured in red stage, astaxanthin can be produced efficiently by irradiation with both blue LED and red LED. There is no particular upper limit to photon flux density, but from the perspective of balancing energy costs and effect, not greater than 3000 μmol/m²/s is preferred, and not greater than 1000 μmol/m²/s is particularly preferred.

The light source in red stage is not particular designated. A nature light and/or a fluorescent lamp may be used, but astaxanthin can be produced at the greatest efficiency in the case that a light source with blue LED of peak wavelength from 420 to 500 nm and red LED of peak wavelength from 620 to 690 nm is used.

The method for recovering astaxanthin from the culture solution is not particularly limited. For example, dried microalga product may be obtained by separating the microalga culture solution containing astaxanthin by solid-liquid separation means such as filtration, centrifugation, and the like to collect microalga cells, and then drying them (natural drying, drum drying, hot air drying, spray drying, freeze drying, and the like). The obtained dried microalga product contains astaxanthin (as a free form) in a concentration from 1 to 10% by mass. The concentration is preferably from 4 to 10% by mass.

A component containing astaxanthin may be obtained by crushing the wet algal or the above dried product containing astaxanthin, and by extracting and recovering astaxanthin. The methods for extracting and recovering astaxanthin are not particularly limited, and methods commonly used by those skilled in the art may be used. For example, astaxanthin is extracted after the dried microalga product is mechanically crushed. Examples of the extraction method include chemical extraction using an organic solvent such as chloroform, hexane, acetone, methanol, ethanol, and the like, and edible oils and fats, or physical extraction by pressing the dried Chlorophyceae product, and the like. Alternatively, it may be extracted or recovered using supercritical extraction. The extraction solvent is distilled out to obtain oil containing astaxanthin.

Methods of LED irradiation of the culture solution include external irradiation in which a culture solution contained in a reactor is irradiated from the outside, and internal irradiation in which LEDs are put into a culture solution contained in a reactor, but either may be used without particular limitation. Note that the value used as photon flux density in the case of external irradiation is that measured on the exterior surface of the container, and in the case of internal irradiation, it is the value at the container surface in contact with the culture solution. Both external irradiation and internal irradiation can be used together.

The microalga culturing apparatus for astaxanthin production is not particularly limited provided that carbon dioxide can be supplied and the culture solution can be irradiated using both a blue LED of peak wavelength from 420 to 500 nm and a red LED of peak wavelength from 620 to 690 nm. For example, in the case of a small scale, a flat culture bottle from 10 to 50 mm thick or a glass tube from approximately 20 to 70 mm in diameter is preferably used. In the case of a large scale, a culture vessel constructed from a plastic bag or a tube or transparent plate made of glass, plastic, and the like, equipped with an illuminator and a stirrer as necessary, may be used. When culturing on a large scale, the light transmission width (diameter, thickness) is preferably not greater than 400 mm, and more preferably not greater than 70 mm. Examples of such a culture vessel include a flat panel culture vessel, tube culture vessel, air dome culture vessel, hollow cylinder culture vessel, internally illuminated tank culture vessel, and the like. In addition, in any case, a tightly sealing container is preferably used. For example, a type in which a tube is coiled around LEDs as disclosed in Japanese Unexamined Patent Application Publication No. 2012-29578A, or a hybrid type of reactor as disclosed in Japanese Unexamined Patent Application Publication No. 2014-39491A may be used.

Types of culturing of astaxanthin include a type placing a vessel outdoors and using sunlight, and a type placing a vessel indoors and using artificial light, and a type using both of the above two types. The method that uses sunlight can produce astaxanthin inexpensively because there are no energy costs, but if the equipment is careless, quality may decrease due to impurities or contaminants. The present invention may be used with either type. Even when using natural light, the effect of the present invention can be obtained by irradiation using both blue LED of peak wavelength from 420 to 500 nm and red LED of peak wavelength from 620 to 690 nm in a condition that photon flux density of blue LED is greater than photon flux density of red LED at least during green stage culturing of microalgae.

When culturing using only artificial light, both blue LED of peak wavelength from 420 to 500 nm and red LED of peak wavelength from 620 to 690 nm are used at least during green stage. Other light sources such as a fluorescent lamp may be used during red stage, but both blue light and red light may also be used during the astaxanthin-producing culturing peroid.

The ratio of photon flux density of blue light to red light during red stage is preferably from 1:1 to 20:1 and more preferably from 1:1 to 5:1, or from 3:2 to 4:1.

Specifically, a culturing in green stage is performed in condition that a ratio of photon flux density of blue LED to red LED is from 2:3 to 5:1. A culturing in red stage is performed in condition that a ratio of photon flux density of blue LED to red LED is from 1:1 to 5:1. Alternatively, a combination as an example includes a culturing in green stage at a ratio from 3:2 to 5:1 and a culturing in red stage at a ratio from 3:2 to 5:1.

The present invention is described in detail below using embodiments, but the present invention is not limited by these embodiments.

In the present invention, astaxanthin quantity was measured by the following method.

Astaxanthin Measurement by HPLC using Luna 3 μm Silica Column

A certain quantity of sample is collected, acetone is added, and the resulting product is crushed. A supernatant is recovered by centrifugal separation. 0.05 M Tris-HCl buffer and cholesterol esterase solution are added to the supernatant and reacted for 45 minutes at 37° C., and astaxanthin is freed. The astaxanthin is extracted with petroleum ether, the solvent is distilled out, and the resulting product is dried. It is dissolved in hexane:acetone=82:18, and used as a sample solution for HPLC. It is measured under the HPLC analysis conditions as following. Because astaxanthin has geometric isomers, astaxanthin content is analyzed based on their peak area.

HPLC Analysis Conditions

Column: Luna 3 μm Silica (2) 100A 150*4.6 mm (Phenomenex Inc.)

Mobile phase solvent: hexane:acetone=82:18 (v/v)

Device start-up method: A-JUNSOU

A-JUNSOU method settings

Injected sample quantity: 20 μL

Mobile phase flow rate: 1.2 mL/min

Column temperature: 30° C.

DAD: 455 nm, 467 nm, 475 nm

Measurement time: 13 min

### Embodiment

### Culturing Haematococcus (Green Stage)

50 mL of culture solution, containing planktonic cells of Haematococcus lacustris strain NIES144 (preserved at the National Institute for Environmental Studies Microbial Culture Collection facility) in a concentration of 500,000 cells/mL, and 700 mL of BG11 modified A medium (Table 1) were separately poured into eight 1L of erlenmeyer flasks made of glass having maximum an outer diameter of 130 mm and a height of 215 mm. The culturing is performed for 5 days at 25° C. while stirring and ventilating with air containing 1% carbon dioxide, under continuous irradiation with a red LED (wavelength is 660 nm) and a blue LED (wavelength is 450 nm) according to the ratio showed in Table 2 at a photon flux density of 50 μmol/m²/s.

### Culturing Haematococcus (Red Stage)

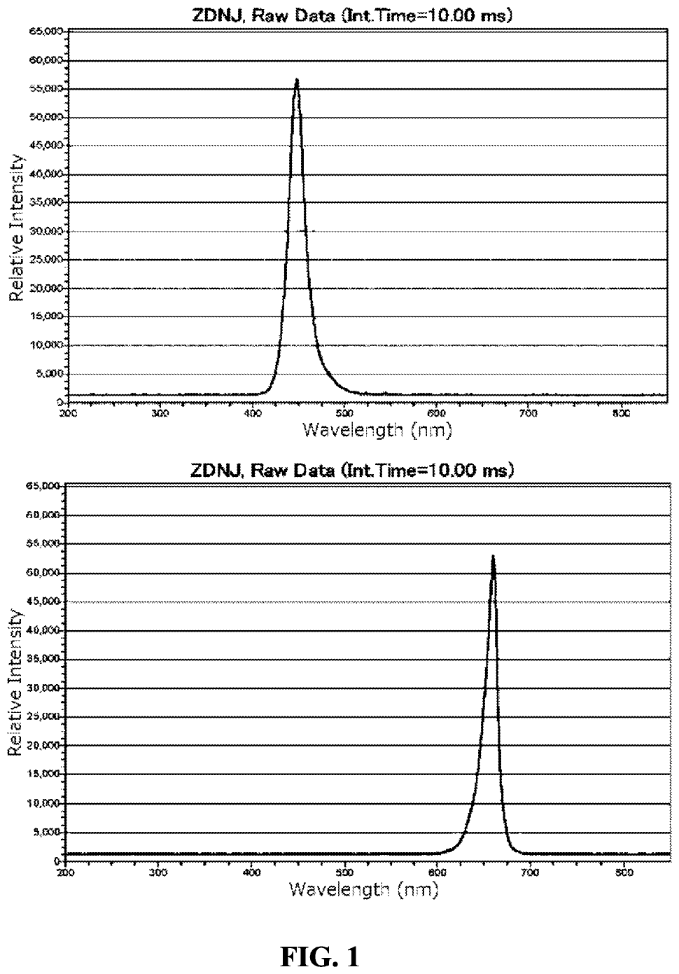

Next, 750mL of culture solution cultured in the 1 L erlenmeyer flasks was separately transferred to eight transparent culture vessels made of glass having an inner diameter of 50 mm and a height of 500 mm. Then, after adding sodium chloride to each culture solution so as to get a concentration of 2 g/L, the culturing is performed at 27° C. while stirring and ventilating with air containing 1% carbon dioxide, under continuous irradiation with a red LED (wavelength is 660 nm) and a blue LED (wavelength is 450 nm) according to the ratio showed in Table 2 at a photon flux density of 300 μmol/m²/s, thereby the production of astaxanthin is performed. The spectra of the blue LED and red LED used in this experiment are shown in FIG. 1. After culturing for 14 days, a dried algal product was obtained by filtration. The dried algal product was weighed, and the dried algal product weight per volume of culture solution was determined. Furthermore, the astaxanthin content in the dried algal product and the astaxanthin production quantity per volume of culture solution were determined by reversed-phase HPLC.

TABLE 1
Ingredient

| | | |
|---|---|---|
| NaNO₃ | 480 | mg |
| CaCl₂•2H₂0 | 21.6 | mg |
| Na₂CO₃ | 12 | mg |
| MgSO₄•7H₂0 | 60 | mg |
| K₂HPO₄ | 30.6 | mg |
| Citric acid | 5.4 | mg |
| Ammonium iron (iii) citrate | 3.6 | mg |
| Micro element solution | 0.3 | mL |
| Vitamin solution | 0.12 | mL |
| Distilled water | 1000 | mL |

Micro element solution

| | | |
|---|---|---|
| ZnSO₄•7H₂0 | 22.2 | mg |
| MnCl₂•4H₂0 | 181 | mg |
| CuSO₄•5H₂O | 7.9 | mg |
| Co(NO₃)₂•6H₂0 | 49 | mg |
| H₃BO₃ | 286 | mg |
| Na₂MoO₄•2H₂0 | 39 | mg |
| Na₂EDTA | 100 | mg |
| Distilled water | 50 | mL |

Vitamin solution

| | | |
|---|---|---|
| Thiamine | 100 | mg |
| B₁₂ | 1.25 | mg |
| Biotin | 12.5 | mg |
| Distilled water | 100 | mL |

The results are shown in Table 2. It can be determined by comparing embodiments 1-5 to comparative examples 1-4 that in green stage, the ratio of photon flux density of blue LED to red LED becomes greater, although dried algal product weight does not become greater, the carotenoids amount in the dried algae becomes more, and as a result, the carotenoids production quantity in culture solution becomes larger.

It has been confirmed that such tendency is irrelevant to light irradiation in red stage. It is more preferable that blue LED and red LED are used together in red stage.

It has been confirmed that astaxanthin content of inner of algae can be increased by irritating with blue LED and red LED simultaneously in green stage during culturing, and as a result, astaxanthin production quantity per volume of culture solution can be increased.

TABLE 2

| | | | | | dried | astaxanthin | |
|---|---|---|---|---|---|---|---|
| | | | | | algal | content | astaxanthin |
| | green stage | | red stage | | product | (in dried algal | production |
| | red LED | blue LED | red LED | blue LED | weight | product) | quantity |
| | μmol/m²/s | μmol/m²/s | μmol/m²/s | μmol/m²/s | g/L | weight % | mg/L |
| comparative | 50 | 0 | 300 | 0 | 3.1 | 2.5 | 78 |
| example 1 | | | | | | | |
| comparative | 36.7 | 13.3 | 300 | 0 | 2.7 | 3.3 | 89 |
| example 2 | | | | | | | |
| embodiment 1 | 13.3 | 36.7 | 300 | 0 | 2.9 | 4.6 | 133 |
| comparative | 50 | 0 | 150 | 150 | 3.2 | 3.8 | 122 |
| example 3 | | | | | | | |
| comparative | 36.7 | 13.3 | 150 | 150 | 3.1 | 4.7 | 146 |
| example 4 | | | | | | | |
| Embodiment 2 | 30 | 20 | 150 | 150 | 3.1 | 5.2 | 161 |
| Embodiment 3 | 25 | 25 | 150 | 150 | 3.1 | 5.7 | 177 |
| Embodiment 4 | 20 | 30 | 150 | 150 | 3.0 | 6.2 | 186 |
| Embodiment 5 | 13.7 | 36.7 | 150 | 150 | 2.9 | 6.7 | 194 |

### INDUSTRIAL APPLICABILITY

By the method of the present invention, the amount of energy usage can be decreased and the astaxanthin production quantity per volume of culture solution can be increased.

1. A method for producing astaxanthin in which astaxanthin is produced in inner of algae by culturing a microalgae, wherein a light irradiation during a green stage culturing of the microalgae is performed by using both a blue LED of peak wavelength from 420 to 500 nm and a red LED of peak wavelength from 620 to 690 nm, and having a ratio of photon flux density of the blue LED to the red LED to be 2:3 to 20:1.

2. The method for producing astaxanthin according to claim 1, wherein the ratio of photon flux density of the blue LED to the red LED is 3:2 to 20:1.

3. The method for producing astaxanthin according to claim 1, wherein the light irradiation using the blue LED and the red LED is continuously performed during the green stage culturing.

4. The method for producing astaxanthin according to claim 1, wherein the photon flux density of the blue LED is from 5 to 200 μmol/m²/s, and the photon flux density of the red LED is from 5 to 200 μmol/m²/s.

5. The method for producing astaxanthin according to claim 1, wherein a light irradiation during a red stage culturing of the microalgae is performed further using both the blue LED of peak wavelength from 420 to 500 nm and the red LED of peak wavelength from 620 to 690 nm.

6. The method for producing astaxanthin according to claim 5, wherein the ratio of photon flux density of the blue LED to the red LED is 1:1 to 20:1 during the red stage culturing of the microalgae.

7. The method for producing astaxanthin according to claim 5, wherein the photon flux density of the blue LED is from 20 to 1000 μmol/m²/s during the red stage culturing, the photon flux density of the red LED is from 20 to 1000 μmol/m²/s during the red stage culturing.

8. The method for producing astaxanthin according to claim 1, wherein the microalgae is Haematococcus genus.
